(19) United States
(12) Patent Application Publication
(10) Pub. No.: US 2011/0196138 A1
(43) Pub. Date: Aug. 11, 2011

(54) SEPARATION OF GLYCYRRHIZIC ACID FROM LICORICE EXTRACT BY ULTRAFILTRATION

(75) Inventors: Leon J. Gorgol, Haddon Heights, NJ (US); Stephen G. Taub, Naples, FL (US); Joseph Olesiewicz, Wilmington, DE (US)

(73) Assignee: Mafco Worldwide Corporation, Camden, NJ (US)

(21) Appl. No.: 12/704,265

(22) Filed: Feb. 11, 2010

(51) Int. Cl. C07H 15/24; B01D 15/00

(52) U.S. Cl. 536/18.1; 422/261

(57) ABSTRACT

A process for separating glycyrrhizic acid from licorice extract feed includes the steps of: providing a licorice extract feed and passing the licorice extract feed through an ultrafiltration device to produce a concentrate and a permeate. The ultrafiltration device contains a membrane that is selected to separate the glycyrrhizic acid from other components of the feed such that at least a substantial percentage of the glycyrrhizic acid is retained in the concentrate.

### TECHNICAL FIELD

The present invention relates to the processing of licorice extract and, more particularly, to a system and process for the separation of glycyrrhizic acid from licorice extract by ultrafiltration without the use of chemicals that generate potentially harmful byproducts.

### BACKGROUND

Licorice extract is the essence of the root of the licorice plant (Glycyrrhiza glabra), which grows wild in portions of central Asia, the Middle East and southeastern Europe. The licorice plant is also known by the names sweetwood and black sugar because licorice extract is very sweet. Licorice extract is light brown in color and is produced by processing the roots of the licorice plant as described below. Although licorice extract is commonly available in liquid form, it also can be concentrated into sticks or blocks, or turned into powder.

There are two principal methods in use currently for making licorice extract. In both methods, the roots of the licorice plants are first harvested and dried, after which they are cleaned by removing physical impurities, such as soil and the like. In one method, the licorice roots are then ground into powder, and the resulting powder can be used as is or mixed with water. In the more commonly used method, the roots are pulped and boiled, and the extract is concentrated by allowing the water to evaporate. If the extract is in dried form, it can be stored indefinitely.

Licorice extract is used in many different types of applications. For example, licorice extract is well known as a candy ingredient, but it is also used in the treatment of various conditions, including sore throats, irritable bowel syndrome and skin diseases such as psoriasis. It also is used in nonfood and nonmedicinal ways. For example, it is a foaming agent used in fire extinguishers.

Glycyrrhizin is the main compound of interest within licorice root. In comparison to normal table sugar (sucrose), glycyrrhizin is generally perceived to be 30-50 times as sweet. Chemically, glycyrrhizin is a titerpenoid saponin glycoside of glycyrrhizic acid. Under hydrolysis, the glycoside loses its sweet taste and is converted to the aglycone glycyrrhetinic acid along with molecules of glucuronic acid.

The conventional practice for separating glycyrrhizic acid from licorice extract uses chemicals that cause a precipitation or separation of the glycyrrhizic acid from the licorice extract. In the case of precipitation, sulfuric acid is generally added to the extract, resulting in a very low pH waste material that is high in COD (chemical oxygen demand) and BOD (biological oxygen demand). This waste material is produced in large quantities and is typically discarded while the precipitate is retained. In cases of separation by ultrafiltration and nanofiltration as generally practiced in the prior art, licorice extract is ammoniated to raise the pH to about 9.8, and the resulting permeate is the retaintant (i.e., the material with the desired glycyrrhizic acid). The concentrate from this process is again discarded. Thus, both of the methods commonly found in the prior art result in the need to discard substantial quantities of potentially harmful material.

There is therefore a need to provide an alternative process for separating glycyrrhizic acid from licorice extract that overcomes the disadvantages that are associated with the conventional chemical-based practices. The present invention satisfies these needs.

### SUMMARY

In accordance with one embodiment of the present invention, a process for separating glycyrrhizic acid from licorice extract includes the steps of: providing a licorice extract feed; and passing the licorice extract feed through an ultrafiltration device that contains a membrane that is selected to separate the glycyrrhizic acid from other components of the feed. The glycyrrhizic acid is present in a concentrate (which represents the retaintant) that is produced by ultrafiltration of the licorice extract feed. The step of providing the licorice extract feed includes the step of preparing a water extraction of licorice root by adding an alpha-amylase enzyme in a prescribed proportion relative to a licorice liquor that includes water.

In a second embodiment of the present invention, a system for separating glycyrrhizic acid from licorice extract includes a source of licorice extract feed and a vessel that is fluidly connected to the source of licorice extract feed for receiving the licorice extract feed. The system also includes an ultrafiltration device that is fluidly connected to the vessel and receives licorice extract feed therefrom. The ultrafiltration device has a membrane that produces a concentrate and a permeate when the licorice extract feed is passed therethrough. The membrane is selected to separate the glycyrrhizic acid from other components of the feed, and the glycyrrhizic acid is present in greater concentrations in the concentrate.

In one exemplary structure consistent with the second embodiment, at least about 85% (by weight) of the glycyrrhizic acid is retained within the concentrate and 15% or less (by weight) of the glycyrrhizic acid is retained within the permeate.

These and other aspects, features and advantages will be apparent from the accompanying Drawing and description of certain embodiments of the invention. One of skill in the relevant art will understand that other embodiments can be used and various processing changes can be made without departing from the scope of the claimed invention.

### BRIEF DESCRIPTION OF THE DRAWING

FIG. 1 is a schematic view of an exemplary system for the separation of glycyrrhizic acid from a licorice extract feed by ultrafiltration in accordance with the present invention.

### DETAILED DESCRIPTION OF CERTAIN EMBODIMENTS OF THE INVENTION

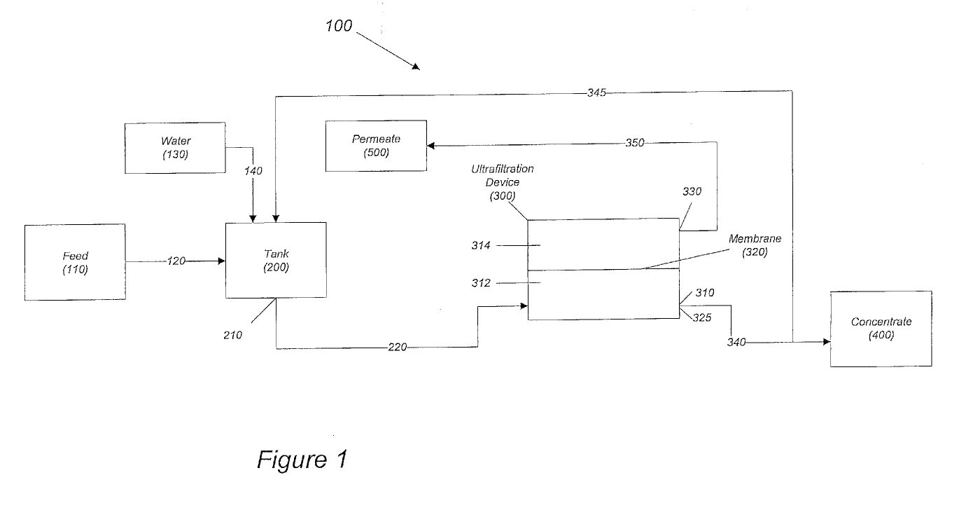

FIG. 1 is a schematic view of an exemplary system 100 for the separation of glycyrrhizic acid from licorice extract by an ultrafiltration process in accordance with the present invention. As described herein, the system 100 overcomes the disadvantages associated with the conventional chemical-based processes and eliminates the potentially significant environmental problems associated with the use of a chemical process to separate glycyrrhizic acid from licorice extract.

The system 100 includes a source 110 of feed, which in the case of the present invention is a licorice extract feed. The system 100 includes a vessel 200 that receives the licorice extract feed from the source 110 by means of a first conduit 120. The first conduit 120 fluidly connects the source 110 to the vessel 200 and can be in the form of a pipe, sluice or the like.

The licorice extract feed can be prepared using any number of conventional processes as described above. For example, the licorice extract feed can be formed by pulping and boiling the licorice roots in water, and then concentrating the extract by allowing a portion of the water to evaporate. The licorice extract feed can thus be in the form of a liquid feed that is delivered to the vessel 200 using conventional techniques such as a pump, sluice or the like.

Any number of different types of structures can be used for the vessel 200. The vessel 200 can be a tank, vessel, receptacle or other type of structure that holds a fixed amount of liquid (e.g., 250 gallons) that is selected depending upon the application. For example and according to one embodiment, the vessel 200 can be a clean-in-place (CIP) stainless steel tank that holds a prescribed volume of liquid based on the overall specifications of the system and the target output rates of the system.

The licorice extract feed can be preprocessed prior to being delivered to the vessel 200. For example, the licorice extract feed can be filtered to eliminate some impurities generally found in a typical licorice extract feed.

A water source 130 can be fluidly connected to the vessel 200 by means of a second conduit 140. The second conduit 140 can be in the form of a pipe, sluice or the like. Additional components (e.g., processing liquids), depending upon the application and the processing specifications, can be delivered to the vessel 200 by means of conduits that permit delivery of these components to the vessel 200. The various ingredients, including the water, feed stock (licorice extract) and optional components (which form a feed stock mixture), can be delivered to one or more different locations within the vessel 200, such as the top, upper or lower side or bottom of the vessel 200.

The vessel 200 includes an outlet 210 that selectively permits the contents of the vessel 200 to be delivered to another location. In the exemplary system 100 shown in FIG. 1, the outlet 210 is fluidly connected to an ultrafiltration device 300 by means comprising a third conduit 220. As with the other conduits, the third conduit 220 can be in the form of a pipe, sluice or the like. The fluid that leaves the vessel 200 and flows out of the outlet 210 is delivered to the ultrafiltration device 300 under the action of a pump, gated sluice acting with gravity or the like. It will also be appreciated that other processing and/or flow-regulating equipment can be provided along the length of the third conduit 220. For example, one or more pumps can be provided for causing the fluid (treated licorice extract) to be delivered to the ultrafiltration device 300. It will also be appreciated that controllable flow regulators can be provided along the length of the third conduit 220 for regulating flow.

The ultrafiltration device 300 is described in more detail herein; however, it generally is a device that performs a separation process that uses at least one membrane with a pore size in the range of 0.001 to 0.1 microns. Typically, an ultrafiltration process removes high molecular-weight substances, colloidal materials, and organic and inorganic polymeric molecules. Low molecular-weight organics and ions, such as sodium, calcium, magnesium chloride, and sulfate, are not removed. Because only high-molecular weight species are removed, the osmotic pressure differential across the membrane surface is relatively low.

Ultrafiltration is a cross-flow separation process. A liquid stream to be treated (feed) flows approximately tangentially along the membrane surface, thereby producing two streams. The stream of liquid that passes through the membrane is called permeate. The type and amount of species left in the permeate will depend on the characteristics of the membrane, the operating conditions, and the quality of feed. The other liquid stream is called the concentrate and gets progressively concentrated due to other components of the feed passing through the membrane to form the permeate that can then be selectively and independently further processed if desired. In the present invention, the concentrate represents the retaintant in that it contains the desirable glycyrrhizic acid.

The ultrafiltration device 300 can therefore be formed of a housing 310 that contains one or more semi-permeable membranes. In FIG. 1, the ultrafiltration membrane 320 is shown as a line that divides the housing 310 into a first section 312 upstream of the membrane 320 and a second section 314 downstream of the membrane 320. It will be understood that the size, shape and other characteristics of the membrane 320 can be selected based on the given application. In addition, it will also be appreciated that the ultrafiltration membrane can be in the form of a plurality of semi-permeable membranes that are arranged relative to one another within the housing 310 so that the incoming feed is separated into concentrate (upstream of the membranes) and permeate (downstream of the membranes).

The third conduit 220 is in fluid communication with the first section 312 of the housing 310 and thus delivers the licorice extract feed to a location that is upstream of the ultrafiltration membrane 320.

As shown in FIG. 1, the housing 310 includes a first outlet 325 and a second outlet 330 with the first outlet being in fluid communication with the first section 312 and the second outlet 330 being in fluid communication with the second section 314. The first outlet 325 is fluidly connected to a first outlet conduit 340 that delivers fluid from the first section 312 to another location, and, in particular, the first outlet conduit 340 delivers the concentrate produced during the ultrafiltration process to another location where it can be further processed as described herein. It will also be appreciated that a portion of the concentrate can be delivered back to the vessel 200 using a return conduit 345 that fluidly connects to the first section 312 via another outlet like first outlet 325 or that branches off of the first outlet conduit 340 and is routed back to and terminates at the vessel 200.

Similarly, the second outlet 330 is fluidly connected to a second outlet conduit 350 that delivers fluid from the second section 314. The second outlet conduit 350 is thus in the form of a permeate conduit for delivering the permeate produced during the ultrafiltration process to another location (e.g., location 500) where it can be further processed.

Preferably, the system 100 is a controllable, programmable system, and, therefore, select components thereof (e.g., the operable parts) are preferably in communication with a master controller or the like. The master controller can thus serve to regulate the flow between the individual devices or stations of the system as well as control other processing parameters such as the temperature of the vessel 200 or the temperature of the liquid flowing along any of the conduits. The system may therefore comprise in select embodiments various controlling or regulating equipment to control or regulate these parameters as desired.

In accordance with the present invention, an ultrafiltration process is used for separating glycyrrhizic acid from the licorice extract feed. In one embodiment the licorice extract feed is formed by preparing a water extraction of licorice root by adding an alpha-amylase enzyme in a prescribed proportion relative to the feed liquor. Alpha-amylase enzyme is an enzyme that hydrolyzes alpha bonds of large alpha-linked polysaccharides, such as starch and glycogen, yielding glucose and maltrose.

For example, the alpha-amylase enzyme can be added in a proportion of about 100 grams per about 5000 dry pounds of licorice extract solution. However, this is merely one embodiment and other effective proportions can be selected in order to form the feed 110. The alpha-amylase enzyme randomly hydrolyzes bonds in the interior of starch, glycogen and their degradation products. The enzyme will continue to hydrolyze the bonds as long as conditions are favorable in that the operating temperature is within a prescribed range and pH is within a prescribed range. For example, the operating temperature should be maintained below 150° F. and pH should be maintained above a prescribed value in order to permit continued hydrolysis of the bonds by the enzyme.

Alpha-amylase enzyme products are commercially available from any number of different sources. For example and in accordance with one embodiment, an exemplary alpha-amylase enzyme is commercially available under the trade name Validase FAA40L. Treatment of the licorice extract solution with an alpha-amylase enzyme produces a feed stock that is identified in FIG. 1 by the character legend 110 that represents a source of the licorice extract feed stock. The licorice extract feed stock flows from the source 110.

As part of the separation process of the present invention, the temperature of the licorice extract feed is maintained at a predetermined temperature in order to provide optimal conditions and optimal separation results. For example, the temperature of the licorice extract feed stock is preferably maintained at or about 130° F. as it flows throughout the system to provide optimal activity for the enzyme and to provide optimal membrane performance since the ultrafiltration membrane can be adversely affected when the feed stock coming into contact therewith has higher temperatures, such as a temperature at or above 135° F. In accordance with one embodiment of the present invention, the licorice extract feed stock has the following characteristics: it contains an average of 3.7% solids, a pH of about 4.8 and a glycyrrhizic acid content of about 13.1% of the total dry solids. However, it will be appreciated that the aforementioned values are merely exemplary and the licorice extract feed stock can have values other than the above ones. For example, the licorice extract feed stock can have the following characteristics: a solid content from about 0.5% to about 8.1%, a pH from about 2.9 to about 6.8 and a glycyrrhizic acid content from about 6.7 to about 21.1% of the total dry solids. Once again, these values are merely exemplary in nature and the licorice extract feed stock can have values that lie outside these ranges.

In one embodiment, the pH of the licorice extract feed stock is the natural pH of the feed stock and is about 3.9 or less since it has been observed that this pH range yielded optimal results. Accordingly and in contrast to conventional separation processes, adjustments to the pH of the licorice extract feed stock are not required.

One of skill in the art will recognize that a pre-filtration stage can be incorporated into the first conduit 120. For example, this pre-filtration stage can be incorporated along the first conduit 120 between the source 110 and the vessel 200. The pre-filtration stage is intended to remove coarse suspended solids and can be formed of any number of different filtration devices, including those employing different types of filtration media. For example, the feed stock can be filtered using a 1000 micron filter, whereby the licorice extract feed stock flowing along the first conduit 120 is fed through the filter in order to remove any coarse solids that may be suspended within it. Other size filters can be also used, in a range of approximately 600 microns through 1000 microns.

The licorice extract feed stock flows into the vessel 200 where it is preferably mixed with other liquids as described herein to form a licorice extract feed stock mixture. The licorice extract feed stock mixture flows from the vessel 200 through the outlet 210 into and through the conduit 220 to the ultrafiltration device 300. The feed stock mixture then enters (under pressure) into the first section 312 of the ultrafiltration device 300 where it flows into contact with the membrane 320. The ultrafiltration device 300 functions in the manner described herein in that it is a selective fractionation by which substances in a solution (i.e., the licorice extract feed stock mixture) are separated on the basis of molecular size. Membranes are used with pore sizes in the range of 0.001-0.1 microns or 1 to 500 kiloDaltons molecular weight cutoff.

In one embodiment, the membrane 320 is a 10 kiloDalton molecular weight cutoff membrane, and certain components of the licorice extract feed stock mixture are conducted across the membrane 320 by pumping the licorice extract feed stock mixture into the ultrafiltration device 300 and into contact with the membrane 320. One exemplary membrane 320 is at least one ultrafiltration membrane that is in the form of a spiral element (spiral wound membrane) and is commercially available from Koch Membrane Systems under the name KMS HFK™-131 Food & Dairy UF Elements. The Koch membrane is a semi-permeable polyethersulfone (PES) membrane on a polyester backing material and has a 10,000 molecular weight cutoff (MWCO). In addition, the Koch membrane has a construction in the form of a sanitary, spiral-wound element with a net outer wrap. A feed spacer can also be provided.

The semi-permeable ultrafiltration membrane is selected so that at least a substantial percentage of the glycyrrhizic acid by weight is separated from other components of the licorice extract feed stock mixture and is retained in the concentrate. In other words, the glycyrrhizic acid content retained in the concentrate can be expressed as a percentage that is retained relative to the glycyrrhizic acid content originally contained in the licorice extract feed stock mixture. The glycyrrhizic acid content is expressed as grams of glycyrrhizic acid per grams of total dry solids content. For example, if the original feed stock mixture contained 10 grams of glycyrrhizic acid, an 85% retention rate of the glycyrrhizic acid in the concentrate would mean that the concentrate has 8.5 grams of glycyrrhizic acid and the permeate has 1.5 grams of glycyrrhizic acid.

As used herein, the term “substantial percentage” refers to a percentage that is at least about 75%. It will be appreciated that in a preferred embodiment, at least a substantial percentage of the glycyrrhizic acid (by weight) is separated and retained in the concentrate; however, the present process and system are not limited to requiring that a substantial percentage of the glycyrrhizic acid be separated and retained in the concentrate (e.g., in certain applications less than 75% of the glycyrrhizic acid (by weight) may be separated and retained in the concentrate). In preferred embodiments, at least 80% of glycyrrhizic acid (by weight) is separated and retained in the concentrate, and more preferably at least 85% (by weight).

As mentioned above, it will be appreciated that the housing 310 of the ultrafiltration device 300 can contain more than one membrane 320. In the exemplary embodiment using the spiral shaped ultrafiltration elements just described, the housing 310 includes a plurality of membranes 320, each in the form of an ultrafiltration spiral shaped element. Other arrangements of membrane 320 employing one or more membranes will be apparent to those skilled in the art.

Moreover, during the ultrafiltration separation process, the temperature of the licorice extract feed stock mixture is maintained at approximately 130° F. using a heat transfer device, such as a shell and tube heat exchanger or other heat exchanging device. Maintaining the licorice extract feed stock mixture at this temperature yields optimal results.

Permeate is collected in the second section 314 of the housing 310 and can be further processed. For example, the permeate can flow out of the outlet 330 and into the conduit 350 and can then be directed to different locations (e.g., location 500) for additional processing, collection, etc.

In addition, a portion of the concentrate that exits the ultrafiltration device 300 and flows along the conduit 340 can be diverted into the conduit 345 for return back to the vessel 200. The concentrate can thus be recycled back to the vessel 200 until the desired concentration level has been reached in the vessel 200. Once this concentration is achieved, the concentrate that is discharged from the first section 312 of the ultrafiltration device 300 can be delivered to concentrate collection location 400. In this case when it is desired to deliver the concentrate to the concentrate collection location 400, the branch 345 can be closed off from the conduit 340 to deliver all of the concentrate removed from the ultrafiltration device 300 to the concentrate collection location 400.

It will be appreciated that liquid flow within the above-described conduits can be achieved using conventional devices, such as circulation pumps or the like, or through gravity. In addition, conventional valves and the like can be used to direct fluid into a respective conduit, such as through a particular branch at a branching point in a conduit.

### Example

Various configurations of the ultrafiltration system of the present invention were examined. According to one embodiment, the solid content in the permeate achieved an average concentration of about 57.8%. The average glycyrrhizic acid content of the permeate was about 14.6% (by weight) for the trial. The resultant concentrate retained approximately 85% of the glycyrrhizic acid (by weight) and about 42% of the solids from the original licorice extract feed without the addition of property-altering chemicals, such as those associated with conventional processing techniques.

It will be appreciated that the ultrafiltration system and process of the present invention overcome the deficiencies of the conventional separation process because chemicals are not used in the present invention, thereby eliminating harmful by-products. In particular and in contrast to conventional separation processes, by not solubilizing the glycyrrhizic acid component through pH adjustment and instead by adding an enzyme, the chemical bonds of the components that makeup the licorice extract feed solution are utilized to separate at least a substantial amount of the glycyrrhizic acid on the concentrate side of the membrane in direct contrast to the conventional processes where the glycyrrhizic acid is collected within the permeate that includes other components as well as a large percentage of glycyrrhizic acid.

While the invention has been described in connection with certain embodiments thereof, the invention is capable of being practiced in other forms and using other materials and structures. Accordingly, the invention is defined by the recitations in the appended claims appended and their equivalents.

What is claimed is:

1. A process for separating glycyrrhizic acid from a licorice extract feed comprising the steps of:
providing a licorice extract feed that includes glycyrrhizic acid and other components;
passing the licorice extract feed across an ultrafiltration device to produce a concentrate and a permeate, the ultrafiltration device containing a membrane that is selected to separate the glycyrrhizic acid from the other components of the licorice extract feed; and
collecting the concentrate that contains the separated glycyrrhizic acid.

2. The process of claim 1, wherein the step of providing the licorice extract feed comprises the step of:
preparing a water extraction of licorice root by adding an alpha-amylase enzyme to a licorice liquor that includes water.

3. The process of claim 2, wherein the enzyme is added in a proportion of about 100 grams of enzyme per about 5000 dry pounds of the licorice liquor.

4. The process of claim 3, wherein the licorice extract feed has the following characteristics: contains about 3.7% solids, a pH of about 4.8 and a glycyrrhizic acid content of about 13.1% (by weight) of the total dry solids.

5. The process of claim 3, wherein the licorice extract feed has the following characteristics: contains between about 0.5% to about 8.1% (by weight) solids, a pH of between about 2.9 to about 6.8 and a glycyrrhizic acid content of about 6.7% to about 21.1% (by weight) of the total dry solids.

6. The process of claim 1, wherein the ultrafiltration membrane has a molecular weight cutoff of about 10 kiloDaltons.

7. The process of claim 1, wherein the ultrafiltration device includes a plurality of ultrafiltration membranes, each ultrafiltration membrane being in the form of a spiral element.

8. The process of claim 1, further including the step of maintaining at a predetermined temperature the licorice extract feed that is introduced into the ultrafiltration device.

9. The process of claim 8, wherein the predetermined temperature is about 130° F.

10. The process of claim 1, further including the steps of:
receiving the licorice extract feed within a vessel prior to delivering the licorice extract feed to the ultrafiltration device; and
recirculating a portion of the concentrate back to the vessel until a desired solids content level is reached in the vessel.

11. The process of claim 1, wherein at least about 85% (by weight) of the glycyrrhizic acid is retained within the concentrate.

12. The process of claim 1, further including the step of maintaining the pH of licorice extract feed that is introduced into the ultrafiltration device at its substantially natural pH.

13. A system for separating glycyrrhizic acid from a licorice extract feed comprising:
a source of the licorice extract feed;
a vessel that is fluidly connected to the source of licorice extract feed for receiving the licorice extract feed; and
an ultrafiltration device that is fluidly connected to the vessel and receives licorice extract feed therefrom, the ultrafiltration device having a membrane that produces a concentrate and a permeate from the licorice extract feed, the membrane being selected to separate the glycyrrhizic acid from other components of the feed such that a substantial percentage (by weight) of the glycyrrhizic acid originally contained in the licorice extract feed is retained in the concentrate.

14. The system of claim 1, wherein the licorice extract feed comprises a water extraction of licorice root produced by adding an alpha-amylase enzyme to a licorice liquor that includes water.

15. The system of claim 14, wherein the enzyme is added in a proportion of about 100 grams of enzyme per about 5000 dry pounds of the licorice liquor.

16. The system of claim 15, wherein the licorice extract feed has the following characteristics: contains about 3.7% (by weight) solids, a pH of about 4.8 and a glycyrrhizic acid content of about 13.1% (by weight) of the total dry solids.

17. The system of claim 13, wherein the licorice extract feed has the following characteristics: contains about 0.5% to about 8.1% (by weight) solids, a pH of between about 2.9 to about 6.8 and a glycyrrhizic acid content of about 6.7% to about 21.1% (by weight) of the total dry solids.

18. The system of claim 13, wherein the ultrafiltration membrane has a molecular weight cutoff of about 10 kiloDaltons.

19. The system of claim 13, wherein the ultrafiltration device includes a plurality of ultrafiltration membranes, each ultrafiltration membrane being in the form of a spiral element.

20. The system of claim 13, further including a device for maintaining the licorice extract feed at a predetermined temperature that is about 130° F.

21. The system of claim 13, wherein the ultrafiltration device has a first outlet through which the concentrate is discharged and a second outlet through which the permeate is discharged, wherein a first outlet conduit is fluidly connected to the first outlet for delivering the concentrate from the ultrafiltration device to another location and a second outlet conduit is fluidly connected to the second outlet for delivering the permeate from the ultrafiltration device to another location.

22. The system of claim 21, wherein the first outlet conduit is fluidly connected to the vessel for recirculating a portion of the concentrate back to the vessel until a desired solids content level is reached in the vessel.

23. The system of claim 13, wherein at least about 85% (by weight) of the glycyrrhizic acid is retained within the concentrate.

24. A process for separating glycyrrhizic acid from a licorice extract feed comprising the steps of:
providing a licorice extract feed that includes glycyrrhizic acid and other components, the licorice extract feed having a natural pH;
passing the licorice extract feed across an ultrafiltration membrane to produce a concentrate and a permeate, the ultrafiltration membrane being selected so as to at least substantially separate the glycyrrhizic acid from the other components of the licorice extract feed such that at least a substantial percentage of the glycyrrhizic acid is retained in the concentrate, wherein the pH of the licorice extract feed that passes across the ultrafiltration membrane is at least substantially the same as the natural pH; and
recirculating at least a portion of the concentrate back to a vessel supplying the device containing the ultrafiltration membrane until a desired solids content level is achieved within the vessel and upon achieving the desired level, the concentrate is collected for further processing.

25. The process of claim 24, wherein at least about 75% (by weight) of the glycyrrhizic acid is separated and retained in the concentrate.

26. The process of claim 24, wherein at least about 80% (by weight) of the glycyrrhizic acid is separated and retained in the concentrate.

27. The process of claim 24, wherein at least about 85% (by weight) of the glycyrrhizic acid is separated and retained in the concentrate.
